SEQUENCE LISTING (34 sequences)

SEQ ID NO: 1 - DNA, 26 bp, synthetic construct
  misc_feature 1..26: note = Probe
  source 1..26: mol_type = other DNA
gataccgctggaaacggctttgtcaa

SEQ ID NO: 2 - DNA, 22 bp, synthetic construct
  misc_feature 1..22: note = Probe
  source 1..22: mol_type = other DNA
cggcacggtcaggttcgtcctt

SEQ ID NO: 3 - DNA, 25 bp, synthetic construct
  misc_feature 1..25: note = Probe
  source 1..25: mol_type = other DNA
attggcatggaagtcacaccttcgg

SEQ ID NO: 4 - DNA, 23 bp, synthetic construct
  misc_feature 1..23: note = Probe
  source 1..23: mol_type = other DNA
ttctgacctgaaggctctgcgcg

SEQ ID NO: 5 - DNA, 24 bp, synthetic construct
  misc_feature 1..24: note = Probe
  source 1..24: mol_type = other DNA
tgctcgggcatcataacggaaagc

SEQ ID NO: 6 - DNA, 24 bp, synthetic construct
  misc_feature 1..24: note = Probe
  source 1..24: mol_type = other DNA
gaagcgcgcgaaatcgaagttgct

SEQ ID NO: 7 - DNA, 24 bp, synthetic construct
  misc_feature 1..24: note = Probe
  source 1..24: mol_type = other DNA
tgctcgggcatcataacggaaagc

SEQ ID NO: 8 - DNA, 28 bp, synthetic construct
  misc_feature 1..28: note = Probe
  source 1..28: mol_type = other DNA
acgccaatgtttatgtaaaccttgcgcc

SEQ ID NO: 9 - DNA, 28 bp, synthetic construct
  misc_feature 1..28: note = Probe
  source 1..28: mol_type = other DNA
ggcgcaaggtttacataaacattggcgt

SEQ ID NO: 10 - DNA, 34 bp, synthetic construct
  misc_feature 1..34: note = Probe
  source 1..34: mol_type = other DNA
cgggcacgccaatgtttatgtaaaccttggcccg

SEQ ID NO: 11 - DNA, 21 bp, Enterococcus faecium
  misc_feature 1..21: note = Primer sense
  source 1..21: mol_type = other DNA
gctttagcaacagcctatcag

SEQ ID NO: 12 - DNA, 20 bp, Enterococcus faecium
  misc_feature 1..20: note = Primer antisense
  source 1..20: mol_type = other DNA
tcgtccgaacrtcttcattt

SEQ ID NO: 13 - DNA, 21 bp, Enterococcus faecalis
  misc_feature 1..21: note = Primer sense
  source 1..21: mol_type = other DNA
gttctagtgtcggaattagca

SEQ ID NO: 14 - DNA, 20 bp, Enterococcus faecalis
  misc_feature 1..20: note = Primer antisense
  source 1..20: mol_type = other DNA
gcttcratcccttgttcaac

SEQ ID NO: 15 - DNA, 23 bp, Escherichia coli
  misc_feature 1..23: note = Primer sense
  source 1..23: mol_type = other DNA
ccgccaatggtaccgcaatccct

SEQ ID NO: 16 - DNA, 25 bp, Escherichia coli
  misc_feature 1..25: note = Primer antisense
  source 1..25: mol_type = other DNA
gctactgccattatattttacggtc

SEQ ID NO: 17 - DNA, 28 bp, Escherichia coli
  misc_feature 1..28: note = Primer sense
  source 1..28: mol_type = other DNA
acgccaatgtttatgtaaaccttgcgcc

SEQ ID NO: 18 - DNA, 24 bp, Escherichia coli
  misc_feature 1..24: note = Primer antisense
  source 1..24: mol_type = other DNA
acatttcacaacacgagctgacga

SEQ ID NO: 19 - DNA, 28 bp, Escherichia coli
  misc_feature 1..28: note = Primer antisense
  source 1..28: mol_type = other DNA
ggcgcaaggtttacataaacattggcgt

SEQ ID NO: 20 - DNA, 24 bp, Influenza A virus
  misc_feature 1..24: note = Primer sense
  source 1..24: mol_type = other DNA
ggaatggctaaagacaagaccaat

SEQ ID NO: 21 - DNA, 18 bp, Influenza A virus
  misc_feature 1..18: note = Primer antisense
  source 1..18: mol_type = other DNA
ctgcagtcctcgctcact

SEQ ID NO: 22 - DNA, 23 bp, Influenza A virus
  misc_feature 1..23: note = Probe
  source 1..23: mol_type = other DNA
ttcacgctcaccgtgcccagtga

SEQ ID NO: 23 - DNA, 21 bp, Influenza B virus
  misc_feature 1..21: note = Primer sense
  source 1..21: mol_type = other DNA
ctcaactcactcttcgagcgt

SEQ ID NO: 24 - DNA, 22 bp, Influenza B virus
  misc_feature 1..22: note = Primer antisense
  source 1..22: mol_type = other DNA
tctggtgataatcggtgctctt

SEQ ID NO: 25 - DNA, 22 bp, Influenza B virus
  misc_feature 1..22: note = Probe
  source 1..22: mol_type = other DNA
tctggtgataatcggtgctctt

SEQ ID NO: 26 - DNA, 20 bp, SARS coronavirus
  misc_feature 1..20: note = Primer sense
  source 1..20: mol_type = other DNA
gcttcagcgttcttcggaat

SEQ ID NO: 27 - DNA, 22 bp, SARS coronavirus
  misc_feature 1..22: note = Primer antisense
  source 1..22: mol_type = other DNA
caatttgatggcacctgtgtag

SEQ ID NO: 28 - DNA, 25 bp, SARS coronavirus
  misc_feature 1..25: note = Probe
  source 1..25: mol_type = other DNA
attggcatggaagtcacaccttcgg

SEQ ID NO: 29 - DNA, 22 bp, Respiratory syncytial virus
  misc_feature 1..22: note = Primer sense
  source 1..22: mol_type = other DNA
tcctaacttctcaagtgtggtc

SEQ ID NO: 30 - DNA, 22 bp, Respiratory syncytial virus
  misc_feature 1..22: note = Primer antisense
  source 1..22: mol_type = other DNA
cttggtttcttggtgtacctct

SEQ ID NO: 31 - DNA, 24 bp, Respiratory syncytial virus
  misc_feature 1..24: note = Probe
  source 1..24: mol_type = other DNA
aggcaatgcagcaggtctaggcat

SEQ ID NO: 32 - DNA, 22 bp, Respiratory syncytial virus
  misc_feature 1..22: note = Primer sense
  source 1..22: mol_type = other DNA
gcaggattgtttatgaatgcct

SEQ ID NO: 33 - DNA, 22 bp, Respiratory syncytial virus
  misc_feature 1..22: note = Primer antisense
  source 1..22: mol_type = other DNA
cacaacttgttccatttctgct

SEQ ID NO: 34 - DNA, 24 bp, Respiratory syncytial virus
  misc_feature 1..24: note = Probe
  source 1..24: mol_type = other DNA
ggtgcagggcaagtgatgttacgg

(19) United States
(12) Patent Application Publication
(10) Pub. No.: US 2025/0012722 A1
(43) Pub. Date: Jan. 9, 2025

(54) DEVICE AND METHOD FOR MULTIPLEXED DETECTION OF NUCLEIC ACID SEQUENCES

(71) Applicant: BFORCURE, Montreuil (FR)

(72) Inventors: Francesco VILLA, Montreuil (FR); Christophe PANNETIER, Montreuil (FR); Maël LE BERRE, Montreuil (FR)

(73) Assignee: BFORCURE, Montreuil (FR)

(21) Appl. No.: 18/293,180

(22) PCT Filed: Jul. 28, 2022
(86) PCT No.: PCT/FR2022/051520

§ 371 Date: Jan. 29, 2024

(30) Foreign Application Priority Data
Jul. 30, 2021 (FR) 2108385

(51) Int. Cl. G01N 21/64; C12Q 1/686; C12Q 1/6876

(52) U.S. Cl. CPC G01N 21/6428; C12Q 1/686; C12Q 1/6876; G01N 2021/6432; G01N 2201/1296

(57) ABSTRACT

A device for multiplexed detection of nucleic acid sequences comprises a thermal cycler arranged to perform a series of thermal cycles with an in vitro nucleic acid extraction reagent containing at least two fluorescence probes combining a quencher and a fluorophore. The fluorescence probes are each arranged to target a distinct nucleic acid sequence and the fluorophores have overlapping fluorescence wavelength ranges. A light sensor is arranged to measure radiation emitted by the fluorophores in the fluorescence wavelength ranges, the radiation emitted by each probe varying depending on whether the latter is in a non-modified state in which the quencher mitigates, or does not substantially mitigate, the fluorescence emission, or in a modified state in which the quencher has an opposite effect on the fluorescence emission. An analyzer is arranged to determine, for each respective fluorescence probe, a value representative of a concentration in a modified state from time signatures derived from the measured fluorescence as a function of time for a given thermal cycle of a reaction mixture comprising one or more probes, which may each be substantially entirely in a non-modified or modified state, and at least two measurements carried out during at least some of the thermal cycles, which values representative of a concentration in a modified state make it possible to qualify the presence of one or more nucleic acid sequences each associated with a distinct fluorescence probe so as to cause the fluorescence probe to change from the non-modified state to the modified state during interaction.

### SEQUENCE LISTING

The instant application contains a Sequence Listing which has been submitted in ASCII format via Patent Center and is hereby incorporated by reference in its entirety. Said ASCII copy, created on Aug. 26, 2024, is named 18293180_2_0.xml and is 42, 664 bytes in size.

The invention relates to the field of the detection of nucleic acid sequences by PCR reaction, or any other in vitro nucleic acid amplification method, and in particular multiplexed detection of nucleic acid sequences by polymerase chain reaction (PCR).

In numerous scenarios from the field of molecular biology, it is necessary to identify with a high level of confidence the presence of one or more DNA or RNA sequences of interest in a sample, and to measure their respective relative concentration, these sequences featuring in a set of sequences of interest.

In these scenarios, it is frequently of interest to be able to test the presence of several sequences simultaneously in the same sample in a single test. The causes of this need may be urgency, the small size of the sample, the low concentration of the sequences of interest in this sample, or the cost of the test (reagents, consumables, automated system occupancy rate, etc.).

When this or this sequences are in insufficient quantities to be characterized, it is conventional to implement an in vitro exponential amplification of these sequences, for example by a polymerase chain reaction (PCR). The aim of this method is to obtain a sufficient quantity of the sought sequences using a mixture of oligonucleotide primer pairs located in these sequences of interest. Several variants of PCR have been developed (for example LATE-PCR, ASPCR). Other in vitro nucleic acid amplification methods are also known, such as the NASBA (nucleic acid sequence-based amplification) method, TMA (transcription-mediated amplification) method, LAMP (loop-mediated isothermal amplification), SDA (strand displacement amplification) and rolling circle amplification.

Various methods are already known for identifying the presence of one or more sequences of interest, after having identified them where applicable. It is possible to carry out sequencing without prerequisites, or targeted sequencing, of the nucleic acids present in the sample. Such methods are generally lengthy and costly and remain limited to certain applications. The most used methods consist of using oligonucleotide probes which can be hybridized on a characteristic portion of their sequence of interest. These probes may be used according to several families of methods:
DNA chip: this consists of fixing the various probes on a solid substrate, for example a 2-dimensional matrix, and locating them by their coordinates in this matrix (principle also referred to as “DNA microarray”). This inhomogeneous phase detection has a substantial manufacturing cost and slow hybridization kinetics which impedes the performances in terms of sensitivity,
liquid chip: this consists of arranging the probes on a liquid chip, see for example the Luminex xMap method published at the address https://www.luminexcorp.com/xmap-technology/. This method is complex to use as it requires a flow cytofluorimeter, and the analysis that it implements is not instantaneous as the beads are analyzed sequentially,
homogeneous phase format: the probes may consist of a single oligonucleotide or the combination of two oligonucleotides, free in solution. The probes are conventionally labeled by means of a fluorophore and a quencher. When a probe is found mixed with a sample which does not contain its target sequence, and the temperature is in the temperature range compatible with its hybridization, the fluorescence of this probe is reduced or suppressed by the spatial proximity of the quencher and the fluorophore. On the other hand, when it interacts with its target sequence, the fluorophore and the quencher are kept at a distance and the probe becomes fluorescent.

The homogeneous phase format may be used according to several variants, of which the most used are detailed hereinafter:
molecular beacon: in this variant, the probe consists of a single oligonucleotide, designed such that the fluorophore and the quencher immediate proximity when the probe is not hybridized and is found at a low temperature. This proximity results most often from the presence of complementary sequences of some bases at the extremities of the specific sequence of the sequence of interest. In solution, these two complementary extremities are hybridized by forming a double helix, which has the effect of moving the fluorophore and the quencher closer together and keeping them at a short distance. When the target sequences are present in the solution and the temperature is below or in the vicinity of the melting point of the duplex formed between the probe and its target, the probes which are hybridized at their target sequences become fluorescent, and
5′ nuclease activity test: in this variant, when the probe is hybridized at its target sequence, the hybridization of a 5′ primer of this probe triggers the polymerization of a new DNA strand. When the polymerase encounters the 5′ extremity of the probe, it cleaves it thanks to its 5′ to 3′ exonuclease activity, which has the effect of irreversibly separating the fluorophore from the quencher. The fluorophore can then express its fluorescence more effectively as the radiation is no longer absorbed by the quencher. The principle of the method making use of the 5′ to 3′ exonuclease activity was described in U.S. Pat. No. 5,210,015 and in a publication by Holland P M et al. “Detection of specific polymerase chain reaction product by utilizing the 5′-3′ exonuclease activity of Thermus aquaticus DNA polymerase”, Proceedings of the National Academy of Sciences August 1991, 88 (16) 7276-7280; DOI: 10.1073/pnas.88.16.7276 PNAS 1991en 1991. In this method, the probe was labeled at 5′ using a radioactive marker. The method was subsequently enhanced by replacing this marker by a fluorophore with the addition of a quencher at 3′ which enables the probe to undergo a change of fluorescence according to whether it is free in solution, or hybridized, or digested (U.S. Pat. No. 5,723,591, probes usable with the Taq-Man™ method).

These formats may themselves be implemented according to the variants such as methods using probes (which may also act as a primer in some of these methods) such as (registered trademark), Amplifluor (registered trademark), MGB Eclipse (registered trademark), Light Upon extension (LUX, registered trademark), Quenching of Unincorporated Amplification 10 Signal Reporters (QUASR) or QZyme (registered trademark) for example.

The homogeneous phase format has numerous advantages including: excellent interaction kinetics of the probes with their respective target sequences, low cost, and excellent performances in terms of sensitivity.

However, carrying out several measurements simultaneously is limited by the ability to separate the signals from the different probes mixed in the same test, which represents a major drawback. Indeed, probes targeting distinct sequences must have emission wavelengths capable of being separated from each other to be able to use them during the same measurement.

Indeed, regardless of the variant selected (molecular beacon or 5′ nuclease activity test or other), it is not possible to use more than one probe per fluorescence channel. The real-time thermal cyclers available on the market measure a fluorescence level for each channel at a single time of each amplification reaction cycle. Hence, if two probes emitting in the sample fluorescence channel are mixed, it is not possible to distinguish their respective contribution respective to the production of the fluorescence signal.

Thus, a real-time thermal cycler capable of measuring 4 fluorescence channels can only detect and discriminate the fluorescence emitted by at most 4 probes and the amplification reactions carried out with this device cannot detect more than 4 target sequences per reaction.

Several methods have been proposed to circumvent this limitation:
discrimination by decoupling excitation and emission: in this method, probes emitting in the same fluorescence channel may be mixed on the condition that they can be excited separately. Some fluorophores may indeed be excited at wavelengths distant from each other, but emit in the same wavelength band. This is in particular the case of fluorophores which emit with a large Stokes offset. Thus, the company Cepheid recently enhanced its GeneXpert (registered trademark) device by extending the number of fluorophores detected from 6 to 10 (see the article by Chakravorty et al. “Detection of Isoniazid-, Fluoroquinolone-, Amikacin-, and Kanamycin-Resistant Tuberculosis in an Automated, Multiplexed 10-Color Assay Suitable for Point-of-Care Use”, 2017, Journal of Clinical Microbiology, 55, 183, published at the address https://doi.org/10.1128/JCM. 01771-16). In the orange emission band for example, 3 fluorophores may thus be discriminated (CF9, excitable in the blue range, CF8, excitable in the green range, CF4, excitable in the yellow range). This method requires an optical module making it possible to analyze the spectral composition of the emitted signal: the excitation in the green range produces signals emitted by the fluorophores CF3, C8 and CF10 in the yellow, orange and infrared ranges, respectively. It furthermore has the drawback of methods using several fluorescence bands, namely optical contamination of a band, or channel, in an adjacent band. These optical contaminations (also known as “crosstalk”) may disturb the analysis of the results in the adjacent bands and be a source of erroneous results, such as false positives for example.
discrimination by the melting curve method: it is known to distinguish probes sharing same the fluorescence emission range by distinguishing the probes by their melting point. For this, a melting curve is produced after initiating the formation of the duplex between the sequence of interest and the oligonucleotide probe(s), by progressively and monotonously increasing the temperature T of the solution, while recording the fluorescence signal F. The maximum of the curve −dF/dT indicates the melting point Tf of the duplex. This melting point makes it possible to ensure that the duplex formed is a homoduplex, or a heteroduplex if this temperature is less than the known temperature of the homoduplex, or even identify, in this case, the mutation(s) at the source of the heteroduplex when the possible mutant sequences are listed in a limitative list and their respective melting points with the probe are known bi-uniquely.

Regarding the melting curve method, some of the methods for detecting sequences of interest described above are incompatible with the necessary conditions for measuring the melting point.

This is in particular the case of the following methods:
amplification with a polymerase having a 5′->3′ exonuclease activity: this is the most frequent case, as with the 5′ nuclease activity test method, the non-mutated polymerases having in general such an activity. This results in a hydrolysis of the 5′ extremity of the probes when it is not inhibited, and the hydrolyzed probe molecules are no longer usable to produce the melting curve.
asymmetric amplification: in the general case of primer-dependent exponential amplifications such as PCR, amplification is referred to as symmetric and these primers are at a stoichiometric concentration, which has the effect that the amplification generates as many sense as antisense strands of DNA. By construction, one of these strands shares the same sequence, or a very similar sequence, as that of the probe, such that this strand may compete with the probe to be hybridized at the target sequence, and suppress the signal emitted by this probe. To circumvent this problem, an asymmetric amplification may be carried out by modifying the ratio of the concentrations of the primers in order to promote the formation of the strand of complementary DNA of the probe, but the amplification then quickly stops being exponential when the primer in small quantity has been used up to become only linear, which lengthens the duration of the test and/or lowers its sensitivity,
multiplex amplification with a universal primer: primers containing a single conserved region in their 5′ region. This makes it possible to reduce the occurrence of primer dimers (Brownie et al “The elimination of primer-dimer accumulation in PCR,” Nucleic acids Res. 1997 Nucleic Acids Res. 1997 Aug. 15; 25 (16): 3235-41. doi: 10.1093/nar/25.16.3235, published at the address https://doi.org/10.1093/nar/25.16.3235.) advantageously and effectively and amplify exponentially a large number of sequences of interest with a single primer pair. On the other hand, it is of course impossible to carry out asymmetric amplification, as the primer used amplifies each of the sense and antisense strands of the sequence of interest.

In the case where the probe is not hydrolyzed, its hybridization may be suppressed or considerably reduced by the presence of the complementary strand formed during the symmetric amplification reaction. This reduction of the signal may result from at least two mechanisms:
competition during hybridization with this complementary strand, which, even at a lower concentration than that of the probe, may be hybridized first and preferentially,
displacement of the probe by the complementary strand, which may be hybridized in homologous 5′ or 3′ regions of the sequence of interest, even after the hybridization thereof. This complementary strand may then displace the probe by toehold-mediated strand displacement according to kinetics dependent in particular on the length of these homologous regions (see the article by Srinivas et al. “On the biophysics and kinetics of toehold-mediated DNA strand displacement,” Nucleic acids research 2013, published at the address https://doi.org/10.1093/nar/gkt801),
discrimination by the TOCE and MuDT methods of the company Seegene: the method hereinabove according to the melting curve is carried out at the end point and therefore does not enable a quantification of the relative concentrations of the sequences of interest if they are simultaneously present. The company Seegene has described two methods for circumventing this limitation. The first, called TOCE (Tagging Oligonucleotide Cleavage & Extension, registered trademark) makes it possible to detect up to 5 probes per fluorescence emission channel using “catcher” fluorescent probes distinguishable by their melting point. The main drawback is the complexity of implementation of this method, which requires adding several oligonucleotides in addition to those used to amplify the sequences of interest. These oligonucleotides are capable of generating artifact products which consume reagents to the detriment of the production of the sought PCR products. The second, called MuDT (Multi Ct values in a single channel, registered trademark), makes it possible to combine up to 3 probes in the same excitation and emission channels. The probes have distinct melting points, separated by several degrees. The PCR amplification is carried out with cycles of which the temperature profile comprises one or more stages during the temperature rise to the melting point around 95° C. The temperature stages are selected between the melting points of the probes, and the fluorescence signal is recorded at each of these stages. By computation, it is possible to determine the contribution of each of the probes to the total fluorescence signal. A significant drawback is that the used thermal cycler must be able to manage this particular cycle profile type, which is incompatible with a rapid PCR wherein it is sought to minimize the temperature change time during cycles. This method is furthermore incompatible with detection in TaqMan mode wherein the probe is hydrolyzed.

To date, the methods which claim to be multiplex are therefore extremely unsatisfactory, either by their complexity, or by their performance, or by their incompatibility with common homogeneous phase formats such as TaqMan probes. For most, they could even be described as multi-fluorescence rather than multiplex, since they especially involve making “parallel” or simultaneous, but not multiplexed, measurements, i.e., of which the signals are combined together.

The invention improves the situation. To this end, it proposes a device for multiplexed detection of nucleic acid sequences comprising a thermal cycler arranged to perform a series of thermal cycles with an in vitro nucleic acid amplification reagent containing at least two fluorescence probes combining a quencher and a fluorophore, said fluorescence probes being arranged to each target a distinct nucleic acid sequence and said fluorophores emitting in overlapping fluorescence wavelengths, a light sensor arranged to measure radiation emitted by said fluorophores in said fluorescence wavelength ranges, the radiation emitted by each probe varying depending on whether the latter is in a non-modified state wherein the quencher attenuates or does not attenuate substantially the fluorescence emission, or in a modified state wherein the quencher has an opposite effect on the fluorescence emission, and an analyzer arranged to determine, for each respective fluorescence probe, a value representative a of concentration in a modified state, from time signatures derived from the measured fluorescence as a function of time for a given thermal cycle of a reaction mixture comprising one or more probes, which may each be substantially entirely in a non-modified or modified state, and at least two measurements carried out during at least some of the thermal cycles, said values representative of a concentration in a modified state making it possible to qualify the presence of one or more nucleic acid sequences each associated with a distinct fluorescence probe so as to cause the fluorescence probe to change from the non-modified state to the modified state when they interact.

This device is particularly advantageous because it makes it possible to detect the presence of a sequence of interest using a characterized fluorescent probe of which a time signature has been previously characterized. Thus, the determination of this time signature makes it possible to use simultaneously probes emitting within the same fluorescence emission band, but the time signatures of which are sufficiently different for their respective contributions to be nonetheless capable of being separated. Advantageously, this time signature also makes it possible to eliminate effectively potential optical contaminations of a fluorescence band with an adjacent band. With this device, the potential optical contaminations may even be used to detect the presence of a sequence of interest.

According to various embodiments, the invention may have one or more the following features:
the thermal cycler is a PCR thermal cycler, and the at least two fluorescence probes are selected from the group comprising TaqMan probes, molecular beacons, dual fluorescence transfer probes, Scorpion probes, Amplifluor probes, MGB Eclipse probes, LUX probes, QUASR probes and QZyme probes,
the in vitro nucleic acid amplification reagent comprises three fluorescence probes combining a quencher and a fluorophore, said fluorescence probes being arranged to each target a distinct nucleic acid sequence and said fluorophores emitting in overlapping fluorescence wavelengths,
the in vitro nucleic acid amplification reagent comprises at least two groups of fluorescence probes combining a quencher and a fluorophore, said fluorescence probes being arranged to each target a distinct nucleic acid sequence and said fluorophores emitting in overlapping fluorescence wavelengths within each group of fluorescence probes
the analyzer is arranged to determine the value representative of the concentration of each fluorescence probe in a modified state by solving a matrix system based on the fluorescence measurements, the time signatures and of the constancy the respective concentration of each fluorescence probe,
the analyzer is arranged to solve one matrix system per group of fluorescence probes,
the analyzer is arranged to use a minimization algorithm, and
the analyzer is arranged to determine the value representative of the concentration of each fluorescence probe in a modified state by applying a gradient descent or using a neural network.

The invention also relates to a method for multiplexed detection of nucleic acid sequences comprising the following operations:
a) carrying out a plurality of thermal cycles on a reagent mixture comprising a sample to be analyzed and an in vitro nucleic acid amplification reagent containing at least two fluorescence probes combining a quencher and a fluorophore, said fluorescence probes being arranged to each target a distinct nucleic acid sequence and said fluorophores emitting in overlapping fluorescence wavelengths,
b) during each thermal cycle, carrying out at least two fluorescence measurements in said fluorescence wavelength ranges, the radiation emitted by each probe varying depending on whether the latter is in a non-modified state wherein the quencher attenuates or does not attenuate substantially the fluorescence emission, or in a modified state wherein the quencher has an opposite effect on the fluorescence emission,
c) determining a value representative of the concentration of each fluorescence probe in a modified state from the measurements of operation b) and from time signatures derived from the measured fluorescence as a function of time for a given thermal cycle of a reaction mixture comprising one or more probes, which may each be substantially entirely in a non-modified or modified state,
said values representative of a concentration in a modified state making it possible to qualify the presence of one or more nucleic acid sequences each associated with a distinct fluorescence probe so as to cause the fluorescence probe to change from the non-modified state to the modified state when they interact.

According to various embodiments, the method may have one or more of the following features:
operation b) comprises carrying out PCR cycles, and the at least two fluorescence probes are selected from the group comprising TaqMan probes, molecular beacons, dual fluorescence transfer probes, Scorpion probes, Amplifluor probes, MGB Eclipse probes, LUX probes, QUASR probes and QZyme probes,
operation b) comprises the use of an in vitro nucleic acid amplification reagent comprising three fluorescence probes combining a quencher and a fluorophore, said fluorescence probes being arranged to each target a distinct nucleic acid sequence and said fluorophores emitting in overlapping fluorescence wavelengths,
operation b) comprises the use of an in vitro nucleic acid amplification reagent comprising at least two groups of fluorescence probes combining a quencher and a fluorophore, said fluorescence probes being arranged to each target a distinct nucleic acid sequence and said fluorophores emitting in overlapping fluorescence wavelengths within each group of fluorescence probes,
operation c) comprises solving a matrix system based on the fluorescence measurements, the time signatures and the constancy of the respective concentration of each fluorescence probe,
operation c) comprises solving one matrix system per group of fluorescence probes, and
operation c) comprises applying a gradient descent or using a neural network.

Further features and advantages of the invention will become more apparent on reading the following description, based on examples given by way of illustration and not limitation, based on the drawings wherein:

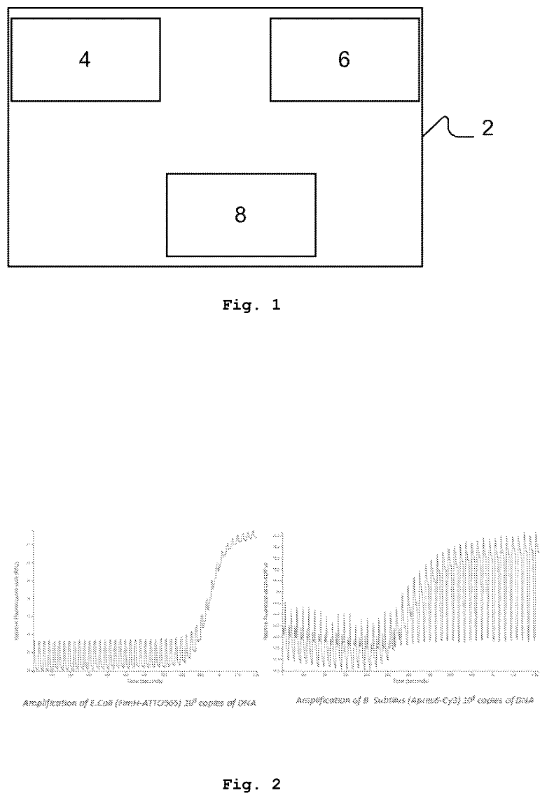

FIG. 1 represents a generic diagram of a device according to the invention,

FIG. 2 shows the fluorescence intensity obtained as a function of time during two PCR reactions using a TaqMan probe labeled with the fluorophore ATTO 565 and fluorophore Cy3 respectively,

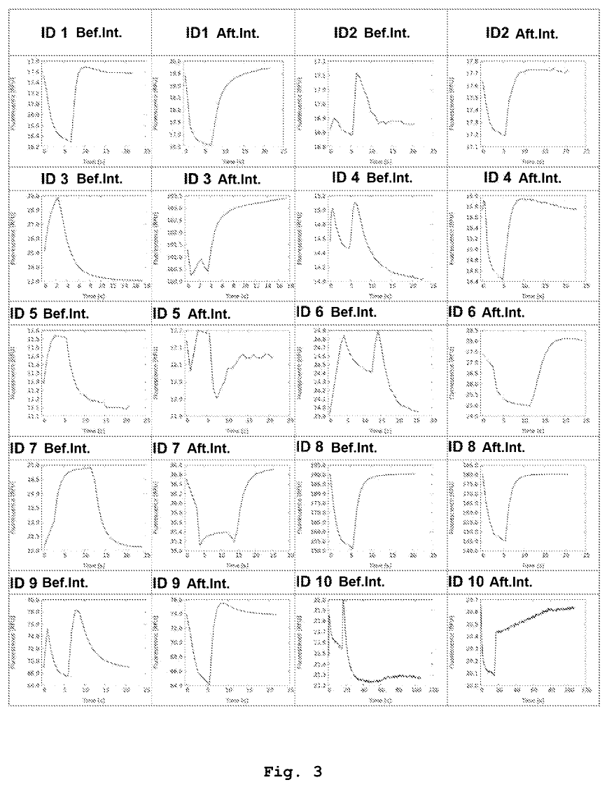

FIG. 3 represents the time signature of a set of probes, with, for each, the profile of the temperature cycle used, the respective components of this signature when the probe has not interacted, and has interacted, with the target sequence for which it is specific,

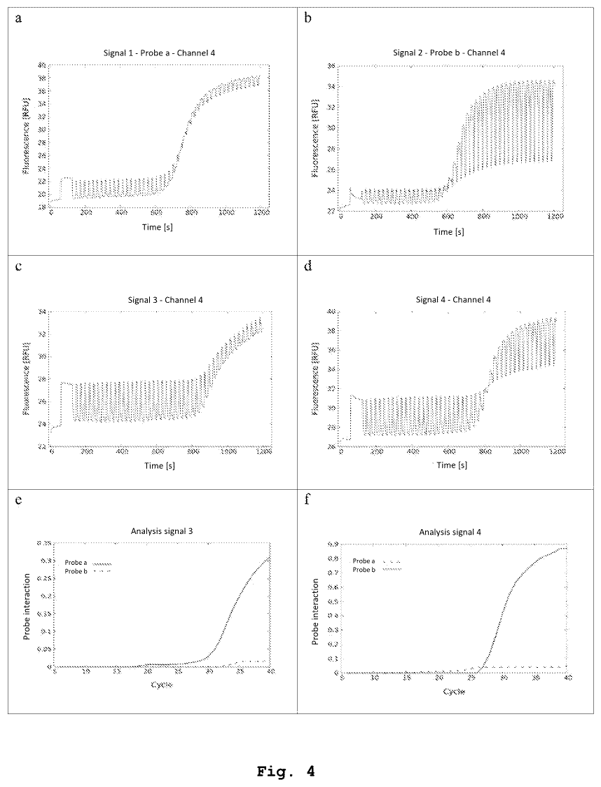

FIG. 4 illustrates the implementation of the method with the device in the specific embodiment of a PCR reaction carried out with two TaqMan probes emitting in the same fluorescence band, comprising the decomposition into an optimal sum of the time signatures as described hereinabove,

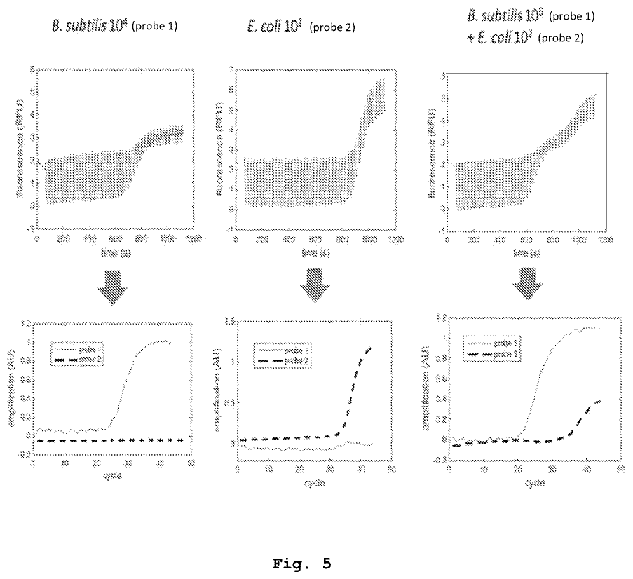

FIG. 5 illustrates the implementation of the method with the device in the specific embodiment of a PCR reaction carried out with two TaqMan probes emitting the same fluorescence in band, comprising the decomposition into an optimal sum of the time signatures as described hereinabove, during a multiplex amplification of two sequences of interest,

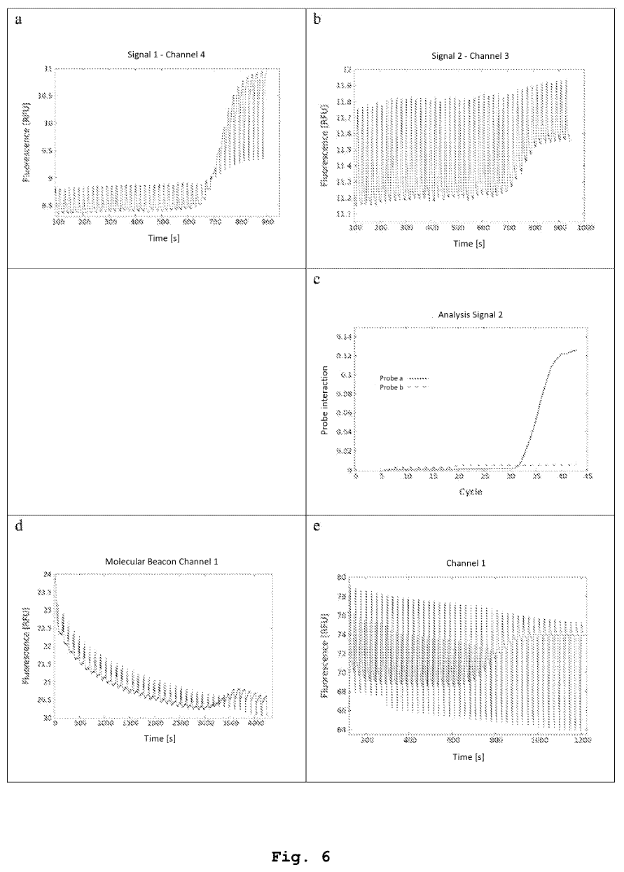

FIG. 6 illustrates the implementation of the method with the device to eliminate the signals of optical contamination of one channel with an adjacent channel, as well as in the specific embodiment of a PCR reaction carried out with molecular beacon and QUASR type probes.

The drawings and the description hereinafter contain, for the most part, elements of a definite nature. Therefore, they may not only serve to make the present invention clearer, but also contribute to its definition, where applicable.

The invention proposes a device capable of carrying out a multiplexed detection of nucleic acid sequences, i.e., using probes comprising one or more fluorophores enabling the identification of genetic sequences by modification of the signal emitted during the interaction of said probes with said sequences (for example TaqMan, Molecular Beacon, FRET probes, etc.), said fluorophores emitting in potentially overlapping fluorescence wavelength ranges.

The interaction of the probe with the target sequence for which it is specific may take several forms according to the detection method used. There are indeed several known methods, such as, without this list being restrictive, real-time PCR detection using TaqMan type probes, molecular beacons, MGB Eclipse probes, dual probes using fluorescence transfer (FRET), or real-time PCR detection using fluorescent primers also serving as Scorpion, Amplifluor, LUX, QUASR or QZyme type probes.

The chemical nature of the probe may also vary according to the methods. It may be rendered in deoxyribonucleic acid (DNA), ribonucleic acid (RNA), or in synthetic nucleic acid analogs such as peptide nucleic acids (PNA), locked nucleic acids (LNA). The probe may include chemical groups modifying its interaction with the genetic sequence for which it is specific, such as a group binding in the minor groove of the double helix (Minor Groove Binder, MGB).

According to the method implemented, the probe may consist of a single oligonucleotide molecule comprising one or more fluorophores and one or more chemical groups having the effect of modulating the intensity of their fluorescence according to the state of the probe, referred to as “quenchers” hereinafter in the description. The probe may also consist of two or more molecules, some comprising one or more fluorophores, the others comprising one or more chemical groups having the effect of modulating the intensity of their fluorescence according to the possible interactions defining the state of the probe. This is for example the case with a QUASR probe comprising two oligonucleotides, or with a dual FRET type fluorescence transfer probe consisting of two oligonucleotides, one carrying at its 3′ extremity a fluorophore capable of being deexcited by transferring its energy to a quencher carried at 5′ of a second oligonucleotide, this quencher itself being capable of being deexcited by emitting light radiation in a different wavelength band from that of the fluorophore.

According to the detection method implemented, the interaction of the probe may consist of a simple hybridization with the genetic sequence for which it is specific, or a hybridization followed by a hydrolysis mediated by an enzymatic activity, or a hybridization followed by an incorporation in an existing DNA strand mediated by the action of a ligase, or in a synthesized DNA strand mediated by the action of a polymerase.

Hereinafter in the text, the term “interact” will be used generically to describe the action of a probe which interacts with the genetic sequence for which it is specific according to one of the methods described hereinabove, and the term “modified by the interaction” to the process whereby the fluorescence characteristics of a probe are modified by its interaction with the genetic sequence for which it is specific according to one of the methods described hereinabove.

The multiplexed detection according to the invention is enabled thanks to the determination of fluorescence time signatures for each type of fluorescent probe used, to the acquisition of two or more fluorescence signals during at least two thermal cycles applied during the amplification reaction, and to the implementation of an algorithm for separating the signals emitted by these probes using these signatures.

Thus, in a specific embodiment of the invention, the device applies to these probes one or more thermal cycles by exciting them optically and recording continuously or in a sampled manner the fluorescence signals emitted in one or more wavelength bands, which makes it possible to carry out the multiplex measurement. When the amplification method comprises a succession of thermal cycles as in the case of PCR, the fluorescence signals are recorded during at least two of these cycles, and preferably, during each cycle. When the amplification method is isothermal, at least two thermal cycles are applied to the sample, one at the start, the other at the end of the reaction, during which the emitted fluorescence signals are recorded.

More specifically, and as represented in FIG. 1, the device 2 according to the invention comprises a thermal cycler 4 capable of applying two or more temperature cycles to an amplification reaction mixture containing at least two fluorescent probes, a light sensor 6 having the capability to carry out at least two fluorescence intensity measurements per cycle in at least one wavelength band, and an analyzer 8 arranged to carry out the measurement of the relative concentration of the different states wherein the different probes are found during the reaction, and to infer therefrom the presence of one or more nucleotide sequences of interest targeted by said probes. In the example described here, the light sensor 6 comprises both the excitation source of the fluorescent probes and the corresponding photodetector. Alternatively, the light sensor 6 could be separated into source and detector. In the example described here, the analyzer 8 is a suitable computer program or code executed on one or more processors. The term processors should be understood to mean any processor adapted for computing texture projection on drawings and voxel-related processing. Such a processor can be embodied in any known manner, in the form of a personal computer microprocessor, a dedicated FPGA or SoC (System on chip) type chip, a computing resource on a grid or in a cloud, a microcontroller, or any other form capable of supplying the computing power required for the embodiment described hereinafter. One or more of these elements can also be embodied in the form of specialized electronic circuits such as an ASIC. A combination of processor and electronic circuits may also be envisaged.

The thermal cycler 4 is capable of applying variable temperatures located in a given range to a sample mixed with an in vitro nucleic acid amplification reagent, this reagent containing at least two fluorescent probes having distinct time signatures. The thermal cycler 4 may repeatedly apply a given temperature profile to this mixture. The analyzer 8 receives or determines the temperature values applied to the sample. In the example described here, these values are sent to the analyzer 8. According to various embodiments, they may be measured in or in contact with the sample, or extrapolated as a function of time. In a preferred embodiment of the invention, the thermal cycler 4 is a fast thermal cycler (i.e., with a temperature change of the order of more than 5° C. per second, and more preferably of more than 15° C. per second). This is enabled thanks to the analyzer 8 according to the invention. Conventionally, fast thermal cyclers are not used to try to carry out multiplex measurements because the information that they supply is not sufficiently precise.

The light sensor 6 is arranged to subject the reaction mixture to a light excitation in one or more wavelength bands in the ultraviolet range and/or in the visible range and/or in the infrared range, and to measure the fluorescence emission intensity resulting from this excitation, in one or more wavelength bands in the ultraviolet range and/or in the visible range and/or in the infrared range, by carrying out one or more point measurements during a defined duration, or a series of point measurements at a given frequency (for example, for 100 ms every 200 ms). These fluorescence measurements as a function of time in the accessible wavelength band(s) are sent to the analyzer 8. In a preferred embodiment, the acquisition periodicity is between 10 ms and 10 s, and even more preferably between 100 ms and 1 s. Thus, unlike the entire prior art, the analyzer 8 can track quasi-continuously the fluorescence response variation throughout the thermal cycles.

In a specific and preferred embodiment of the method according to the invention, the PCR reaction is used to detect and discriminate the presence or absence of a genetic target in combination, the PCR reagent containing a set of probes combining a quencher and a fluorophore (such as TaqMan probes for example), said fluorescent sondes using distinct fluorophores which can emit in a common wavelength band or range.

Thanks to the prior knowledge of the time signature of each of the probes, the analyzer 8 is capable of measuring unambiguously, for each probe present in the reagent, the fluorescence signal modification resulting from the interaction between the probe and the amplification products (or amplicons) obtained from the genetic sequence for which it is specific, independently of each other, and thus attaining a multiplexing level of 2n, or even 3n or 4n, with a device according to the invention capable of measuring the fluorescence signal in n wavelength bands simultaneously.

This method makes it possible to detect that a sequence of interest is present in a sample, not by merely observing an increase in fluorescence at low temperature (60° C. for example) over time as done conventionally during a real-time in vitro nucleic acid amplification analysis, but by observing the change of the fluorescence profile measured by the light sensor 6 during a thermal cycle. FIG. 2 shows two fluorescence curves recorded with the device 2, in the same fluorescence channel, but during PCR reaction, wherein the probes used are functionalized with two different fluorophores (ATTO 565 and Cy3). These curves show that the fluorescence profiles as a function of time during a cycle vary, according to the type of probe, and according to whether the cycle is located at the start or at the end of amplification.

In a specific embodiment of the method according to the invention, the thermal cycle is a PCR cycle and comprises the temperature increase from a low temperature to a high temperature, the low temperature being between 55 and 70° C., preferably between 58 and 65° C. and the high temperature being between 8° and 100° C., preferably between 9° and 98° C., followed by the temperature decrease from the high temperature to the low temperature.

In the present description, the thermal cycles are described generically as comprising the temperature increase from 60 to 95° C., followed by the temperature decrease to 60° C. These values are particularly adapted to the implementation of a fast PCR reaction. It goes without saying that the values of these ranges (60° C., 95° C. and 60° C.) could be modified to adapt to specific thermal cycles used during a PCR amplification with for example the use of 3 temperatures instead of 2, or even thermal cycles added during an isothermal amplification according to a temperature profile compatible with the thermal stability of the reagents used.

In a specific embodiment of the method according to the invention, the signal modification results from the hydrolysis of the probe(s) in the case of TaqMan probes cleaved by a polymerase having an exonuclease activity.

Furthermore, the device 2 may be used to determine the time signature of each of the probes present in the reaction mixture.

### General Principle of the Invention

The general principle implemented by the invention is based on the detection of the modification of the signal of the fluorescence emitted by a probe according to whether it has interacted or not with the nucleotide sequence of interest for which it is specific. The Applicant's work made it possible to identify 1) that this fluorescence signal depends generally on the temperature via several more or less well-known physical phenomena which take place concomitantly, of which the main ones will be cited hereinafter; 2) that the variation of the fluorescence signal of this probe with the temperature may by measured by applying a temperature cycle to the sample containing the probe(s): 3) that the fluorescence curve obtained itself varies according to whether the probe has interacted or not with the nucleotide sequence for which it is specific; 3) that this variation of the signal variation during a cycle is specific to the probe and forms a signature which may be used to detect, at the time of application of a thermal cycle, the fraction of this probe which is found to have interacted with the target sequence for which it is specific; 4) that the determination of this fraction at at least two selected times of the amplification reaction can make it possible to infer therefrom the presence or absence, or the initial concentration the determination of a threshold cycle, in the amplification reaction mixture, of the nucleotide sequence of interest for which the probe is specific.

Note that in most cases, the fluorescence level of a probe interacting with a specific nucleotide sequence varies thanks to the modification of the distance between a fluorophore and a quencher (case of TaqMan, Molecular Beacon, FRET, MGB Eclipse probes, and primers acting as Scorpion, Amplifluor, LUX, QUASR probes, etc.), this fluorescence variation may depend on the temperature in several ways.

Firstly, the variation of the quantum yield of the fluorescence of the fluorophore used, which lowers as the temperature increases. For example, the yield of rhodamine B decreases uniformly with the temperature passing from 0.8 to 10° C. to 0.3 to 60° C. (see the article by Kubin et al. “Fluorescence quantum yields of some rhodamine dyes”, Journal of Luminescence 1983, doi.org/10.1016/0022-2313(82)90045-X). In this specific case, this decrease is essentially due to the increase in dynamic quenching with temperature (see the technical note by Amaoutakis 2016, “Quenching of fluorescence with temperature”, Technical note from EDINBURGH INSTRUMENTS published at the address https://www.edinst.com/wp-content/uploads/2018/10/TN_27-Quenching-of-Fluorescence-with-Temperature.pdf). This variation of the quantum yield with temperature in turn varies with the type of fluorophore. Thus, the variation of the quantum fluorescence yield with temperature of two fluorophores emitting in the same wavelength range may differ from one fluorophore to another.

Then, the change of configuration of the probe, which modifies the distance between the fluorophore and the quencher, or the location of the fluorophore or the quencher (for example, the fluorophore of a probe may be in the vicinity of the quencher of another probe or of the quenchers of several probes) affects the fluorescence signal emitted by the fluorophore. More generally, the amplification reaction mixture containing the fluorescent probes and the other reagents is regulated by intramolecular and intermolecular interactions between the different chemical species. The oligonucleotides in solution may adopt different configurations as a function of the temperature of the stage of the amplification reaction (the presence at increasing concentration of complementary sequences to the probes generated during the amplification reaction). As a first approximation, for an intact probe, different scenarios are possible at each time of the amplification reaction:
1) the probe interacts with a complementary sequence generated during the amplification reaction,
2) the probe does not interact with any complementary sequence, or
3) the probe is bonded to complex structures, with one or more oligonucleotides in solution bonded to each other.

The probe may therefore adopt different states, for example different configurations, according to whether it is in one or the other of these scenarios.

The effectiveness of the quencher in quenching the fluorescence emitted by the fluorophore is a third factor influencing the intensity of the fluorescence of the probe with temperature.

Finally, the state of the probe may also be an important factor: In the TaqMan method, when the probe is hybridized on its target sequence and has been hydrolyzed in the detection mode via the 5′ to 3′ exonuclease activity of the polymerase, the fluorophore is cleaved irreversibly from the probe and its fluorescence is no longer by the proximity of the quencher. The interaction of the probe with the nucleotide sequence for which it is specific thus results in a change of state wherein the fluorophore of the probe is no longer molecularly bonded to the quencher.

The Applicant discovered experimentally that the variation laws of each of these phenomena with temperature may themselves vary substantially according to the structure of the probe and in particular the nature of the fluorophore, the sequence of the probe and the nature of the quencher, as well as the proximity with another probe. According to the parameters, the combination of these different laws may produce an overall law very variable from one probe to another. In particular, the effect of temperature on the quantum yield of the fluorophore and that of temperature on the configuration of the probe have contributions of opposite signs and of different time constants.

The Applicant's work enabled it to identify that, for a given temperature profile, this curve forms the first component of a time signature.

When a nucleotide sequence complementary to one of the probes is present in a sample at a certain concentration and one of the molecules carrying these sequence interacts with a molecule of this specific probe of this sequence, the law of variation of the intensity of fluorescence as a function of temperature of this probe may be modified, whether because the probe adopts another configuration on account of its hybridization state (as is the case for Molecular Beacon probes), because a component of the probe is in the vicinity of another components of the probe having a disruptive effect (as is the case of FRET type dual probes or Scorpion primers) or a polymerase having a 5′ to 3′ exonuclease activity is present in the sample, and the fluorophore of the probes which are hybridized with these complementary sequence is cleaved by this exonuclease (as is the case for TaqMan probes), the law of variation of the fluorescence as a function of temperature of this probe is modified.

The Applicant's work enabled it to identify that, for a given temperature profile, this variation forms a second component of the time signature which substitutes the first component of the signature.

By analyzing and comparing the fluorescence signals recorded during a given temperature cycle before and after the interactions resulting from the presence of the amplification product, it therefore becomes possible to compute, for each probe and at the time when the thermal cycle is applied, its fraction that was modified by this interaction, and thus infer therefrom the quantity of the amplification product obtained from this specific sequence present in the sample thanks to these two time signature components.

During the first cycles or first times of an amplification reaction wherein a certain quantity of a sequence of interest is found, the molecules of the probe for which it is specific are not modified significantly by the presence of an amplification product obtained from this sequence of interest (except in the specific case of a high initial concentration of this sequence of interest), and the fluorescence signal recorded originates solely from the time signature components of the probe not modified by the interaction with the sequence for which it is specific. After a certain number of cycles, an increasing fraction of the probe is modified following the interaction with the amplification product obtained from the sequence of interest, and the recorded signal is a linear combination of the respective time signature components of the probe non-modified and modified by the interaction with the amplification products obtained from the sequence of interest.

If several fluorescent probes emitting in the same fluorescence band are mixed and recording is performed continuously, with a sufficient sampling frequency, the fluorescence intensity emitted by this mixture of probes during one or more cycles (before and after modification of these probes by the presence of the amplified targeted sequence(s)), it is possible, thanks to their respective time signatures, to separate at each cycle the contributions to the signal of each of the probes by determining for each probe the fraction which is found in non-modified form and the fraction which is found in modified form by the interaction with the nucleotide sequence for which it is specific.

When probes emit mostly in fluorescence bands that are not common but adjacent as in the case of numerous organic fluorophores, the method makes it possible in particular to eliminate optical contaminations from one band to the other (“crosstalk”) or any other fluorescence variation not originating from the modification of the probe by the interaction with the sequence for which it is specific. In the prior art, these optical contaminations are eliminated conventionally by applying a linear correction using a square matrix of which the non-diagonal terms are dependent on the identity of the fluorophores used in a multiplex kit. For example, with a thermal cycler having 4 fluorescence detection channels, the user enters the identity of the fluorophores used in their kit. This indication makes it possible to select an optical contamination correction matrix adapted to this combination of fluorophores.

In the method according to the invention, such a correction with a matrix is no longer possible because several fluorophores emitting predominantly in channel A may contaminate, at distinct rates, the signal in an adjacent channel B. Thus, the rate of contamination generated by all of these fluorophores in channel B will depend on the amplification or not of each of the sequences of interest detected in channel A. In the method according to the invention, for a probe emitting predominantly in a channel A, the time signatures of this probe in the adjacent channel(s) are determined or known. Moreover, these signatures are used to determine precisely their contribution to the signal in this or these adjacent channels. It is thus possible to subtract this contribution to the signal to eliminate the optical contaminations effectively.

The characterization of the fluorescent probes by determining their respective time signatures, in their non-modified form and their modified form therefore makes it possible to compute, at the time when a thermal cycle is applied to the reaction mixture, the fraction of a probe modified after interaction with the sequence for which it is specific. If this thermal cycle is applied repeatedly during an in vitro nucleic acid amplification reaction, and the fraction which is found in modified form at each cycle is inferred by computing, this fraction being indicative of the quantity of the amplification products obtained from the nucleotide sequence for which the probe is specific, the curve of the variation of this fraction may be established as a function of time, or the number of cycles in the case of a PCR type reaction, and a threshold cycle (Ct) inferred therefrom which makes it possible subsequently to infer therefrom the quantity of the sequence of interest for which the probe is specific (for example, by transferring the value of this threshold cycle to a calibration curve).

In the specific embodiment where the amplification reaction is the PCR reaction and at least one of the probes is a TaqMan probe, the first component of the time signature of this probe consists of the variation of the intensity of its fluorescence during the thermal cycle in the absence of hybridization of this probe with its complementary sequence. The second component of its signature is the variation of the intensity of its fluorescence during the thermal cycle after its hydrolysis, i.e., after cleaving the fluorophore (or quencher) from the rest of the probe.

In the specific embodiment where the amplification reaction is PCR and at least one of the probes used is a molecular beacon, the first component of the time signature of this probe consists of the variation of the intensity of its fluorescence during the thermal cycle in the absence of hybridization of this probe with its complementary sequence. The second component of its signature is the variation of the intensity of its fluorescence during the thermal cycle in the presence of its complementary sequence. Advantageously, molecular beacon probes have the advantage of providing a superior fluorescence contrast between their different folding states.

In the specific embodiment where the amplification reaction is PCR and at least one of the probes is a FRET type probe, the first component of the time signature of this probe consists of the variation of the intensity of the fluorescence of the oligonucleotide carrying the acceptor fluorophore during the thermal cycle in the absence of hybridization of this oligonucleotide with its complementary sequence. The second component of its signature is the variation of the intensity of the fluorescence during the thermal cycle in the presence of its complementary sequence, of which the FRET signal when the two oligonucleotides of the probe are hybridized adjacently on their common target sequence. Advantageously, as FRET probes may be more flexible to characterize mutations or to limit the number of oligonucleotides sequences in the PCR amplification reagent via the use of several FRET probes on the same amplicon, their use makes it possible to limit the number of oligonucleotides present, therefore their interaction, therefore facilitate the increase in multiplexing.

### Determination of the Time Signature of a Given Probe

For each sequence of interest, fluorescent oligonucleotide probe is designed comprising a complementary oligonucleotide sequence characteristic of a portion of said sequence of interest, and of which is known, or failing that of which is determined, for a given fluorescence channel, the time signature (Snm(t), Sm(t)) comprising two components consisting of the curve of the fluorescence intensity as a function of time for a fixed thermal cycle, where no probe is modified by the interaction with its sequence of interest, and where all the molecules of the probe are modified by the interaction with its sequence of interest respectively.

This probe may be of Taqman, Molecular Beacon, dual fluorescence transfer probe, Scorpion, Amplifluor, MGB Eclipse, LUX, QUASR or QZyme type without this list being restrictive.

As seen above, for a given fluorescence channel, the time signature comprises a component for the non-modified form of the probe, hereinafter Snm(t), and a component for the modified form of the probe following the interaction with its sequence of interest, hereinafter Sm(t).

The components of the time signature (Snm(t), Sm(t)) may be determined respectively by directly applying the thermal cycle to the probe in the buffer used for the reaction, respectively in its non-modified form or in its form modified by the interaction with its sequence of interest. Alternatively, the time signature may also be obtained by determining the fluorescence profile of the probe in its two forms as a function of the temperature (SnmT(T), SmT(T)), and by applying the function obtained to the function describing the temperature profile PT(t) (mathematically, Snm is the compound of the functions SnmT and PT, i.e., SnmToPT; similarly, Sm is the compound of the functions SmT and PT, i.e., SmToPT). Alternatively, the components of the time signature of the probe in its non-modified form or in its modified form may be determined indirectly by subtracting between the fluorescence intensity profile of a combination of the probe and other probes or fluorescent sources and the fluorescence intensity profile of the same combination in the absence of the probe for which the signature is to be determined. Advantageously, a combination of time signatures may be used rather than the time signature of a probe alone to determine the presence of a probe directly or indirectly. For example, the signature of the combination of several probes and the signature of the combination of these probes in the absence of one of the probes may be used to determine the absence of the same probe, or the signature of the combination of several probes and the signature of the combination of the same probes in the absence of one of the probes and in the presence of the disappearance of the absent probe to determine the transformation of the same probe (for example, the absence of the signature of a TaqMan probe and the presence of the signature of the hydrolyzed probe). The use of a combination of signatures avoids having to characterize each probe individually and makes it possible to avoid any interaction effects between the probes in the fluorescence signal. As a general rule, the signal of any combination of probes may be used according to the invention as a direct or indirect marker (by combining several signals) of the reagent composition status.

FIG. 3 shows time signature examples for TaqMan or QUASR type probes using various fluorophores and quenchers: the non-modified time signature component and the modified time signature component following the interaction with the sequence for which it is specific.

TABLE 1

| | | Fluorophore | Quencher | |
|---|---|---|---|---|
| Id | Type | (5′) | (3′) | Sequence |
| 1 | TaqMan | FAM | BHQ1 | FAM- |
| | | | | GATACCGCTGGAAACGGCTTTGTCAA- |
| | | | | BHQ1 |
| 2 | TaqMan | ATTO488 | BHQ1 | ATTO488- |
| | | | | CGGCACGGTCAGGTTCGTCCTT-BHQ1 |
| 3 | TaqMan | ATTO565 | BHQ2 | ATTO565- |
| | | | | ATTGGCATGGAAGTCACACCTTCGG- |
| | | | | BHQ1 |
| 4 | TaqMan | CY3 | BHQ2 | CY3-TTCTGACCTGAAGGCTCTGCGCG- |
| | | | | BHQ2 |
| 5 | TaqMan | Texas RED | BHQ2 | TexasRED- |
| | | | | TGCTCGGGCATCATAACGGAAAGC-BHQ2 |
| 6 | TaqMan | CY5 | BHQ2 | CY5-GAAGCGCGCGAAATCGAAGTTGCT- |
| | | | | BHQ2 |
| 7 | TaqMan | ATTO647N | BHQ2 | ATTO647N- |
| | | | | TGCTCGGGCATCATAACGGAAAGC-BHQ2 |
| 8 | QASR | FAM | — | FAM- |
| | | | | ACGCCAATGTTTATGTAAACCTTGCGCC |
| 9 | QASR | FAM (1) | BHQ1 (2) | 1: FAM- |
| | | | | ACGCCAATGTTTATGTAAACCTTGCGCC |
| | | | | 2: |
| | | | | GGCGCAAGGTTTACATAAACTTGGCGT- |
| | | | | BHQ1 |
| 10 | Mol. | FAM | BHQ1 | FAM- |
| | beacon | | | CGGGCACGCCAATGTTTATGTAAACCTTG |
| | | | | GCCCG-BHQ1 |

FIG. 3 references the “IDs” of Table 1 above and represents for each probe the non-modified time signature component (“Bef.Int.”) and the modified time signature component (“Aft.Int”) following the interaction with the sequence for which it is specific.

According to a preferred embodiment, probes emitting in the same fluorescence wavelength range are selected such that their time signatures (in their non-modified state and in their modified state) are sufficiently distinct to be capable of being discriminated.

### Reaction and Determination of Probe Concentration

Once a set of probes has been selected, a reaction mix comprising the sample containing the sequences of interest of unknown number and concentration is made with the latter. Besides the sequences of interest, this reaction mix comprises, for each sequence of interest sought, the fluorescent probe selected and for which the time signature has been characterized directly or indirectly as described above, a set of primers compatible with its probe and capable of amplifying the sequence of interest, reagents and enzymes required to carry out the amplification reaction. In the case of a PCR detection, it consists in particular of dNTPs and at least one heat-resistant polymerase (in the variant wherein the probes are TaqMan type, the polymerase has a 5′ to 3′ exonuclease activity). The reaction mix may also comprise a reverse transcriptase if at least one of the sequences of interest is in RNA.

In the variant wherein the amplification method is PCR and the probes are TaqMan or Molecular Beacon type, the primers are non-fluorescent and located on the sequence of interest on either side of the probe. In the sub-variant wherein the probes are TaqMan type, the polymerase furthermore has a 5′ to 3′ exonuclease activity.

In the variant wherein the amplification method is PCR and a probe is Scorpion, Amplifluor, LUX or QZyme type, this probe also acts as one of the two primers.

Then, the thermal cycler 4 subjects the reaction mix to one or more cycles of the temperature profile, this number being capable of varying from 1 to 40 typically, where applicable after having performed a cycle enabling the reverse transcription of RNA sequences of interest and/or a cycle enabling high-temperature polymerase activation (“hot start”). During these cycles, the fluorescent probe(s) are excited and the light sensor 6 records the resulting fluorescence continuously on the fluorescence channels corresponding to the fluorescence wavelength ranges of each probe. The sampling frequency of the light sensor 6 is selected such that at least two fluorescence measurements are made per cycle, preferably at least one in the denaturing step and at least one in the hybridization step (which may also be the elongation step in the case of a PCR reaction) preferably at a higher frequency making it possible to cover intermediate temperatures which enables a superior discrimination power.

The analyzer 8 is then invoked to process the fluorescence signals measured in each fluorescence channel by the light sensor 6, by decomposing them, for each channel and at each cycle in which the signals were acquired, in the form of a linear combination of the time signature components of the probes in this channel or one of their combinations. This makes it possible to determine, at each cycle or before and after the appearance of the amplification signal, the fraction of each probe which is found in non-modified form, and that which is found in modified form. This determination may be carried out qualitatively by visual interpretation of the time signatures or quantitatively by a numeric computation with a suitable algorithm.

Several algorithms may be used to extract the time signature provided that they allow the decomposition of measured signatures into a sum of characteristic signatures of the presence of a probe in its modified or non-modified form or of any combinations of presence or absence of several probes in their modified or non-modified form. The algorithm used may for example be an algorithm searching for each cycle, the weights of the linear combination of the characteristics signatures optimally approximating the signatures measured for each cycle. These weights may be directly or indirectly representative of the modification of a probe following its interaction with the sequence for which it is specific, resulting from the amplification of this sequence by the amplification reaction. The following section details an implementation of this algorithm.

At the k-th thermal cycle applied during the amplification reaction, and in a given fluorescence channel, the fluorescence signal Fk(t) measured by the light sensor 6 may be defined as follows:

$$\\ \begin{matrix}{F}_{k} ( t ) = {\sum }_{i = 1}^{n} {c}_{i , k}^{{}_{} m} {f}_{i}^{{}_{} h} ( t ) + {\sum }_{i = 1}^{n} ( {c}_{i} - {c}_{i , k}^{{}_{} m} ) {f}_{i}^{{}_{} nm} ( t ) & [ Equation 1 ]\end{matrix}$$

Where:
t is the time during the temperature cycle,
n is the total number of probes in the reaction mixture,
cᵢ is the quantity (or concentration) of probes of index i (this quantity is known),
c_{i,k}^{m} is the quantity (or concentration) of probe of index i present in modified form at the start of the k-th cycle of the amplification reaction,
fᵢ^{m}(t) represents the time signature of the probe of index i when it is modified, and
fᵢ^{nm}(t) represents the time signature of the probe of index i when it is non-modified.

By carrying out the change of variable

$$\\ \begin{matrix}{y}_{k} ( t ) = {F}_{k} ( t ) - {\sum }_{i = 1}^{n} {c}_{i} {f}_{i}^{{}_{} nm} ( t ) & [ Equation 2 ]\end{matrix}$$

The following equation is obtained

$$\\ \begin{matrix}{y}_{k} ( t ) = {\sum }_{i = 1}^{n} {c}_{i , k}^{{}_{} m} ( {f}_{i}^{{}_{} m} ( t ) - {f}_{i}^{{}_{} nm} ( t ) ) & [ Equation 3 ]\end{matrix}$$

As the quantity Σᵢ₌₁ⁿcᵢfᵢ^{nm}(t) is the sum of the components of the signatures of the non-modified probes for cycle k, and is known because cᵢ is known and independent of cycle k, Equation 3 defines a matrix system which associates the fluorescence signal measured at the concentrations of each probe of index i for cycle k.

These coefficients are those which cancel the matrix equation

$$\\ \begin{matrix}[ \begin{matrix}{f}_{1}^{{}_{} h} ( {t}_{1} ) - {f}_{1}^{{}_{} nh} ( {t}_{1} ) & ⋯ & {f}_{n}^{{}_{} h} ( {t}_{1} ) - {f}_{n}^{{}_{} nh} ( {t}_{1} ) \\ ⋮ & ⋱ & ⋮ \\ {f}_{1}^{{}_{} h} ( {t}_{m} ) - {f}_{1}^{{}_{} nh} ( {t}_{m} ) & ⋯ & {f}_{n}^{{}_{} h} ( {t}_{m} ) - {f}_{n}^{{}_{} nh} ( {t}_{m} )\end{matrix} ] [ \begin{matrix}{c}_{1 , k}^{{}_{} h} \\ ⋮ \\ {c}_{n , k}^{{}_{} h}\end{matrix} ] - [ \begin{matrix}{y}_{k} ( {t}_{1} ) \\ ⋮ \\ {y}_{k} ( {t}_{m} )\end{matrix} ] = 0 & [ Equation 4 ]\end{matrix}$$

Where the tᵢ values correspond to the sampling times by the light sensor 6 during the application of the thermal cycle by the thermal cycler 4. It is recalled that the thermal cycle comprises the step of heating from the lower temperature to the upper temperature and the step of cooling from the upper temperature to the lower temperature.

As the measurements are not perfect, the c_{i,k}^{h} values may be selected to minimize the difference. Numerous algorithms make it possible to carry out this optimization, for example least squares.

Thus, it is apparent that, thanks to the time signatures, it is possible to carry out multiplexing/demultiplexing of degree 2, 3, 4, 5 or more, considering that it is possible to clearly define the time signatures of each of the probes. This multiplexing/demultiplexing is carried out in all the fluorescence channels of the light sensor 6, which makes it possible to identify the optical contributions of a probe in the channel wherein it emits predominantly, but also in the adjacent channels where applicable.

The generalized implementation of the algorithm described above necessitates characterizing the probes individually, which necessitates the use of indirect measurements (the signature of the probes when they are not combined) capable of introducing artifacts dans in the determination of the vector c.

Advantageously, it may be possible to carry out a similar computation using more direct signatures (for example, the signatures of the combined probes with or without the modification of a probe) by carrying out a change of space by a linear transformation U:

$$\\ \begin{matrix}[ \begin{matrix}{y}_{k} ( {t}_{1} ) \\ ⋮ \\ {y}_{k} ( {t}_{m} )\end{matrix} ] {\to }_{U} [ \begin{matrix}{y}_{k}^{{}_{} ′} ( {t}_{1} ) \\ ⋮ \\ {y}_{k}^{{}_{} ′} ( {t}_{m} )\end{matrix} ] & [ Equation 5 ]\end{matrix}$$

such that

$$\\ [ \begin{matrix}{y}_{k}^{{}_{} ′} ( {t}_{1} ) \\ ⋮ \\ {y}_{k}^{{}_{} ′} ( {t}_{m} )\end{matrix} ] = U [ \begin{matrix}{y}_{k} ( {t}_{1} ) \\ ⋮ \\ {y}_{k} ( {t}_{m} )\end{matrix} ]$$

such that U makes it possible to solve the same problem of determining the vector c using more directly measurable time signatures.

Advantageously, only some parts of the time signature may be used, to increase the precision of the decomposition. Indeed, some phenomena such as the variation of the quantity of modified probe during the elongation phase of the PCR cycle resulting from the polymerase activity or the non-reproducibility of the temperature change profile at short times may alter the shape of the signature at certain times and it may be preferable not to take these parts of the signature into account for the determination of the quantities of modified probe. In some modes of implementation, the significance of each time of the time signature may be weighted for the computation in order to optimize its impact on the computation result.

Alternatively, the analyzer 8 could function purely numerically and not as a matrix, using for example a gradient descent algorithm to optimize the residual approximation error of the signature measured by the linear combination of characteristic signatures, or contain lookup table type tables, or use a neural network trained to return the concentrations of each probe as a function of the input measurement signal.

Advantageously, the algorithm may use a more complex model than the linear combination of the signatures, accounting for example for the influence of the variation of the quantity of modified probe within a cycle or the progression over the cycles of the quantity of modified probes which may be constrained by hypotheses such as the shape of the amplification curve. The algorithm may then take the form of an optimization computation constrained by the minimization of an energy function or take the form of a neutral network trained to learn specific parameters of an amplification based on the succession of signatures.

FIG. 5 illustrates the implementation in the specific embodiment of a PCR reaction with two TaqMan probes. It shows the extraction of the respective concentrations of each hydrolyzed probe by decomposition into an optimal sum of the time signatures as described above. The top curves show the raw fluorescence signal during the PCR with the same mix containing 2 TaqMan probes emitting in the same band, specific respectively for two bacterial genomes which are used pure (the first on the left and the second in the center), or mixed in different quantities (right). The bottom curves show the respective concentrations of the 2 hydrolyzed probes computed at each cycle according to an extraction with the same parameters when only the first probe is hydrolyzed during the extraction (left graphs), only the second probe is cleaved (center graphs) and when the 2 probes are cleaved according to different kinetics during the PCR (right graphs). The bottom right graph shows the capability of the device according to the invention to extract the amplification signals of these two distinct targets (here the sequences belonging to the genome of B. subtilis and E. coli) at different initial concentrations using probes sharing the same fluorescence signal.

### EMBODIMENT EXAMPLES

### Example 1: Identification of the Presence of a Nucleotide Sequence of Interest in a Multiplex PCR Analysis Using Two TaqMan Probes Functionalized with Fluorophores Both Emitting Mostly in the Band Above 650 nm

The example hereinafter shows how the method and the device according to the invention make it possible to identify the presence of a target sequence of interest among two target sequences using a multiplex PCR analysis using two TaqMan probes functionalized with fluorophores both emitting in the band above 650 nm. These probes, each specific for one of the sequences of interest, emit a fluorescence signal measured in the same channel of the device, but have a different time signature. The use of the demultiplexing algorithm combined with the knowledge of these signatures makes it possible to identify the amplification of one target or the other.

The primers are specific for amplifying two sequences of the [D-alaline-D-alanine ligase] gene. For E. faecium, the sequences of the sense and antisense primer are GCTTTAGCAACAGCCTATCAG and TCGTCCGAACRTCTTCATTT, for E. faecalis, the sequences of the sense and antisense primer are GTTCTAGTGTCGGAATTAGCA and GCTTCRATCCCTTGTTCAAC. Two TaqMan type fluorescent probes are functionalized at the 5′ extremity with the fluorophore ATTO647N for E. faecalis (sequence TGCTCGGGCATCATAACGGAAAGC, see line ID 7 of Table 1) and CY5 for E. faecium (sequence GAAGCGCGCGAAATCGAAGTTGCT, see line ID of Table 1). The two probes are functionalized at the 3′ extremity with the quencher BHQ2. We identify the two probes as “Probe a” and “Probe b” respectively.

In a first step, two PCR reactions are carried out with each primer pair combined with the probe and the DNA of the sequence of the corresponding bacteria, in order to characterize the signature of each of the two probes separately:
in a first PCR, 10⁵ E. faecalis genomes (extracted and quantified DNA) are amplified and the probe specific for E. faecalis is added to the PCR mixture at the concentration of 0.1 μM. The two primer pairs are added to the PCR mixture at the concentration of 0.5 μM for each primer. The signal measured in channel 4 of Chronos DX is referenced as “Signal 1”.
in a second PCR, 10⁶ E. faecium genomes (extracted and quantified DNA) are amplified and only the E. faecium probe is added to the PCR mixture at the concentration of 0.2 μM. The two primer pairs are added to the PCR mixture at the concentration of 0.5 μM for each primer. The signal measured in channel 4 of Chronos DX is referenced as “Signal 2”.

In these two PCR reactions, the reaction mixture is composed of a buffer (Tris-HCL, KCl 25 mM, (NH₄)₂SO₄ 16 mM, MgCl₂ 4.5 mM) and the amplification is carried out using 3U of Taq DNA Polymerase and 0.2 mM of dNTPs. The PCR protocol used consists of: denaturing/activation of the polymerase carried out for one minute at 95° C. and 45 PCR cycles (composed of two phases: denaturing for 3 seconds at 95° C. and hybridization/extension for 15 seconds at 60° C.). The fluorescence intensity measurements are performed every 100 ms.

FIG. 4 shows the signal measured on channel 4 during the two experiments (FIG. 4 a: signal “1” for probe “a” and FIG. 4 b: signal “2” for probe “b”).

Both time signature components of each of the probes are extracted from these signals (signatures shown in lines 6 and 7 of FIG. 3).

After defining the signatures of each of these two probes, multiplex amplification reactions are carried out with a mixture of the reagents using the method according to the invention. The two probes are added to the PCR mixture respectively at the concentration of 0.1 μM (probe a, E. faecalis) and 0.2 μM (probe b, E. faecium). Both primer pairs are added to the PCR mixture at the concentration of 0.5 μM for each primer:
in a first PCR, 10³ genomes of E. faecalis (extracted and quantified DNA) are amplified. The signal measured in channel 4 of Chronos DX is referenced as “Signal 3”.
in a second PCR, 10⁵ genomes of E. faecium (extracted and quantified DNA) are amplified. The signal measured in channel 4 of Chronos DX is referenced as “Signal 4”.

In FIG. 4, the signal measured on channel 4 during these two multiplex PCR reactions is shown (FIG. 4 c: signal “3” and FIG. 4 d: signal “4”).

Signals “3” and “4” are then demultiplexed using the algorithm according to the invention and the time signatures of each probe, by computing, for each cycle of each multiplex PCR reaction, the representative value of the concentration of each of the modified probes. The result of this demultiplexing is shown in FIG. 4 for each of the multiplex amplification reactions (FIG. 4 e: signal 3 analysis, FIG. 4 b: signal 4 analysis). Observing the curves of the representative values of the concentration of each modified probe, as a function of the PCR reaction cycle, makes it possible to identify unambiguously the nature of the sequence of interest that was actually present in each of the two reactions.

### Example 2: Identification of the Presence of a Mixture of Two Nucleotide Sequences of Interest in a Multiplex PCR Analysis Using Two TaqMan Probes Functionalized with Fluorophores Both Emitting Mostly in the 575-615 nm Band

In this example, it is sought to ascertain the respective quantities of two genomic DNA sequences of two B. subtilis and E. coli bacteria using TaqMan type probes emitting mostly in the 575-615 nm fluorescence band. The probe specific for E. coli is labeled with the fluorophore ATTO 565 and the probe specific for B. subtilis is labeled with Cy3.

After determining the time signatures of these two probes as in the example above, a series of 3 PCR amplifications is carried out using a reaction mixture comprising both primer pairs and both probes required to amplify both nucleotide sequences of interest.

In the first reaction, 10⁴ B. subtilis genomes are added; in the second reaction, 10² E. coli genomes are added; in the third reaction, 10⁴ B. subtilis genomes and 10² E. coli genomes are added. The reactions are carried out using the device 2. The fluorescence signals recorded in channel 3 during each cycle of each of the PCR reactions are shown in the top line of FIG. 5.

The algorithm 8 is then used to demultiplex these curves and, for each amplification reaction and for each probe, a curve is obtained showing the representative value of the modified probe according to the cycle number. It is observed that it is possible to detect from these demultiplexed curves the nature of the nucleotide sequence(s) of interest present in the reaction mixtures. For each of these 6 curves, it is possible to measure a threshold cycle (Ct), that may be transferred to a standard curve linking the cycle Ct and the logarithm of the initial quantity of molecules of each of the sequences of interest in order to quantify the quantity of each of the nucleotide sequences of interest present in the mixture.

### Example 3: Correction of the Optical Contamination Induced by the Fluorescence Signal Produced During a PCR During the Hydrolysis of a TaqMan Probe “a” for the Detection of the Presence of Human and Viral Sequences of Interest

The PCR experiment is carried out using primers and probes specific for the viral genome of SARS-CoV-2 and for an endogenous control gene in human nasopharynx epithelial cells. The kit contains probes for the detection of 3 target sequences in 3 optical channels of the device 2: channels 1 and 3 for two targets of SARS-CoV-2 coronavirus, channel 4 for the endogenous control target. In this experiment, the human DNA contained in a sample is detected by PCR amplification of the control sequence of interest with a pair of primers. The fluorescence signal is generated by a probe functionalized with a fluorophore at the 5′ extremity and a quencher at the 3′ extremity, this fluorophore emits a fluorescence signal in a wavelength band detectable in optical channel number 4 of the device 2. This probe is references as Probe “a” and its signal as “Signal 1”, shown in FIG. 6 a.

On account of the spread of the emission spectrum of probe a, the signal generated by the probe is also detected in optical channel number 3 of the device 2. This signal is referenced as “Signal 2”. This contamination generates a visible increase in the signal generated by the probe used for the detection of a target sequence of SARS-CoV-2 present in channel number 3, which could be interpreted incorrectly as the presence of this target sequence in the sample. This probe is referenced as probe “b.” The signal 2 is represented in FIG. 6 b.

The PCR protocol used in this experiment consists of: reverse transcription carried out for 30 seconds at 50° C., denaturing of the nucleic acids and activation of the polymerase carried out for one minute at 95° C., followed by 45 PCR cycles (composed of two phases: denaturing 3 for seconds at 95° C. and hybridization/extension for 15 seconds at 60° C.). The sampling period of the fluorescence measurement is 100 ms throughout the PCR.

To eliminate the optical contamination signal, the method according to the invention, comprising the following steps, is applied:
Determining the time signature of the 2 probes present in the kit detected in optical channels 3 and 4 of the device 2. These signatures are obtained from a PCR with detection of 1.2 μl of the positive amplification control supplied with the kit. These signatures are identified as signals 3 and 4.
Using the algorithm of the analyzer 8 according to the invention to demultiplex the signals present in signal 2. The demultiplexing result is shown in FIG. 6 c. It is observed that the curve corresponding to the signal of probe b is perfectly flat, which indicates that the sequence of interest of the viral genome of SARS-CoV-2 is absent from the sample.

### Example 4: Detection of a Sequence of Interest by Processing the Time Signature of a “Molecular Beacon” Type Fluorescent Probe Used in a Multiplex PCR Reaction

In this example, the method and the device according to the invention are used to detect the presence of a nucleotide sequence of interest in a sample thanks to a PCR amplification using “Molecular Beacon” (MB, Molecular Beacon) type probes. In the method using one or more molecular beacons, these probes are not cleaved during the reaction, unlike the TaqMan type probes. In this aim, the polymerase selected is devoid of 5′ to 3′ exonuclease activity, and is replaced by a strand displacement (SD) activity.

In this example, a PCR is carried out with the following protocol: initial denaturing/activation of the polymerase for 30 seconds at 92° C. and 40 PCR cycles (composed of two phases: denaturing for 5 seconds at 92° C. and hybridization/extension for 30 seconds at 62° C.).

The reaction mixture is composed of a buffer (SD polymerase reaction buffer) by adding MgCl₂ at the final concentration of 3 mM and the amplification is carried out using 50 of SD Polymerase HotStart and 0.2 mM of dNTPs. Primers at the concentration of 0.2 μM (sense primer, sequence CCGCCAATGGTACCGCAATCCCT) and 2 μM (antisense primer, sequence GCTACTGCCATTATATTTTACGGTC) are used to amplify a target sequence of the fimH E. coli gene (10⁴ bacteria added directly in the PCR after bacterial culture in LB medium and quantification). The molecular beacon type fluorescent probe (sequence FAM-CGGGCACGCCAATGTTTATGTAAACCTTGGCCCG-BHQ1) is at the concentration of 0.03 μM.

The signal measured in channel 1 of the device 2 during the PCR reaction of 45 cycles is presented in FIG. 6 d. From this curve, both time signature components of this molecular beacon type probe are extracted. These components are shown in FIG. 3 with reference to ID 10.

This signature is then used to demultiplex the signals recorded during multiplex PCR reactions which uses this probe to detect the presence of the corresponding nucleotide sequence among other sequences.

### Example 5: Determination of the Time Signature of a Probe According to the QUASR Method Implemented in a PCR Reaction

The invention may be used to analyze the fluorescence curves during PCR amplification using the QUASR (Quenching of Unincorporated Amplification Signal Reporters) system.

In this example, a PCR is carried out with the following protocol: initial denaturing/activation of the polymerase for one minute at 95° C. and 45 PCR cycles (composed of deux phases: denaturing for 3 seconds at 95° C. and hybridization/extension for 15 seconds at 60° C.). The sampling period of the fluorescence measurement is 200 ms throughout the PCR.

The reaction mixture is composed of a buffer (Tris-HCL, KCl 25 mM, (NH₄)₂SO₄ 16 mM, MgCl₂ 4.5 mM) and the amplification is carried out using 30 of SuperHotTaq DNA polymerase and 0.2 mM of dNTPs. “Sense” and “antisense” primers of respective sequence FAM-ACGCCAATGTTTATGTAAACCTTGCGCC and ACATTTCACAACACGAGCTGACGA at the concentration of 0.5 μM are used to amplify 10⁵ E. coli genomes (extracted and diluted after bacterial culture in LB medium). A complementary oligonucleotide of the “sense” primer GGCGCAAGGTTTACATAAACATTGGCGT-BHQ1 is added to the reaction mixture at the concentration of 0.3 μM.

The fluorescence intensity curve as a function of time is measured during 45 PCR cycles and illustrated in FIG. 6 e. This curve makes it possible to extract both time signature components of the QUASR type probe used. This signature is shown in FIG. 3 with the ID 9.

This signature is then used to demultiplex the signals recorded during multiplex PCR reactions which uses this probe to detect the presence of the corresponding nucleotide sequence among other sequences.

### Example 6: Detection of 5 Nucleotide Sequences in an Influenza Respiratory Virus a, B, Syncytial Respiratory Virus A and B, and SARS-CoV-2 Syndromic Kit Using a Pentaplex PCR

The method and the device described in the present invention make it possible to detect the presence of 5 viruses using a pentaplex PCR carried out using five primer pairs and five probes for the detection of 5 targets using only two optical channels of the device (channels 3 and 4).

The primers and the probes used are specific for the following targets:

Channel 3:
Target 1-Influenza A:
sense primer
GGAATGGCTAAAGACAAGACCAAT
antisense primer
CTGCAGTCCTCGCTCACT
probe
ATTO565-TTCACGCTCACCGTGCCCAGTGA-BHQ2
Target 2-Influenza B:
sense primer
CTCAACTCACTCTTCGAGCGT
antisense primer
TCTGGTGATAATCGGTGCTCTT
probe
CY3-TCTGGTGATAATCGGTGCTCTT-BHQ2
Target 3-SARS-CoV-2:
sense primer
GCTTCAGCGTTCTTCGGAAT
antisense primer
CAATTTGATGGCACCIGTGTAG
probe
Alexa Fluor 568-ATTGGCATGGAAGTCACACCTTCGG-BHQ2
Channel 4:
Target 4-RSVB:
sense primer
TCCTAACTTCTCAAGTGTGGTC
antisense primer
CTTGGTTTCTTGGTGTACCTCT
probe
ATTO647N-AGGCAATGCAGCAGGTCTAGGCAT-BHQ2
Target 5-RSV type A:
sense primer
GCAGGATTGTTTATGAATGCCT
antisense primer
CACAACTTGTTCCATTTCTGCT
probe
Cy5-GGTGCAGGGCAAGTGATGTTACGG-BHQ2

The primers are added to the mixture at a concentration between 0.1 and 0.6 μM; the probes are added to the reaction at a concentration between 0.1 and 0.5M. The RT-PCR mixture is composed of a buffer (Tris-HCL, KCl 25 mM, (NH₄)₂SO₄ 16 mM, MgCl₂ 4.5 mM) and the amplification is carried out using between 3 and 100 of TAQ polymerase (with 5′ exonuclease activity) and 30 of WarmStart reverse transcriptase. The PCR protocol used consists of: reverse transcription carried out for one minute at 60° C., denaturing/activation of the polymerase carried out for one minute at 95° C. and 45 PCR cycles (composed of two phases: denaturing for 3 seconds at 95° C. and hybridization/extension for 15 seconds at 60° C.). The fluorescence intensity measurements are performed every 100 ms.

5 PCR reactions comprising all the probes except one are carried out to determine the time signatures of the probes.

These time signatures are then used to demultiplex the signals recorded during the pentaplex PCR reactions to detect, in a single reaction, the presence of any sequence among the 5 nucleotide sequences of interest.

Thus, it is apparent that, thanks to the time signatures, it is possible to carry out a multiplexing/demultiplexing of degree 2, 3, 4, 5 or more, considering that it is possible to clearly define the time signatures of each of the probes.

This multiplexed detection capability of nucleic acid sequences opens up substantial possibilities, such as for example the possibility of carrying out simultaneous detection of numerous point genetic mutations of the SARS-CoV-2 coronavirus genome in a clinical sample, which makes it possible to rapidly identify the variant present in this sample, a more rapid and less costly alternative than sequencing of the viral genome present in this sample.

1. A device for multiplexed detection of nucleic acid sequences comprising:
a thermal cycler configured to perform a series of thermal cycles with an in vitro nucleic acid amplification reagent containing at least two fluorescence probes combining a quencher and a fluorophore, said fluorescence probes being configured to each target a distinct nucleic acid sequence and said fluorophores emitting in overlapping fluorescence wavelengths;
a light sensor configured to measure radiation emitted by said fluorophores in said fluorescence wavelength ranges, the radiation emitted by each probe varying depending on whether the probe is in a non-modified state in which the quencher attenuates or does not attenuate substantially the fluorescence emission, or in a modified state in which the quencher has an opposite effect on the fluorescence emission; and
an analyzer configured to determine, for each respective fluorescence probe, a value representative of a concentration in a modified state, from time signatures derived from the measured fluorescence as a function of time for a given thermal cycle of a reaction mixture comprising one or more probes, which may each be substantially entirely in a non-modified or modified state, and at least two measurements carried out during at least some of the thermal cycles, said values representative of a concentration in a modified state making it possible to qualify the presence of one or more nucleic acid sequences each associated with a distinct fluorescence probe so as to cause the fluorescence probe to change from the non-modified state to the modified state when they interact.

2. The device according to claim 1, wherein the thermal cycler is a PCR thermal cycler, and the at least two fluorescence probes are selected from the group comprising TaqMan probes, molecular beacons, dual fluorescence transfer probes, Scorpion probes, Amplifluor probes, MGB Eclipse probes, LUX probes, QUASR probes and QZyme probes.

3. The device according to claim 1, wherein the in vitro nucleic acid amplification reagent comprises three fluorescence probes combining a quencher and a fluorophore, said fluorescence probes being configured to each target a distinct nucleic acid sequence and said fluorophores emitting in overlapping fluorescence wavelengths.

4. The device according to claim 1, wherein the in vitro nucleic acid amplification reagent comprises at least two groups of fluorescence probes combining a quencher and a fluorophore, said fluorescence probes being configured to each target a distinct nucleic acid sequence and said fluorophores emitting in overlapping fluorescence wavelengths within each group of fluorescence probes.

5. The device according to claim 1, wherein the analyzer is configured to determine the value representative of the concentration of each fluorescence in a modified state by solving a matrix system based on the fluorescence measurements, the time signatures and the constancy of the respective concentration of each fluorescence probe.

6. The device according to claim 4, wherein the analyzer is configured to solve one matrix system per group of fluorescence probes.

7. The device according to claim 5, wherein the analyzer is configured to use a minimization algorithm.

8. The device according to claim 1, wherein the analyzer is configured to determine the value representative of the concentration of each fluorescence probe in a modified state by applying a gradient descent or using a neural network.

9. A method for multiplexed detection of nucleic acid sequences comprising the following operations:
a) carrying out a plurality of thermal cycles on a reagent mixture comprising a sample to be analyzed and an in vitro nucleic acid amplification reagent containing at least two fluorescence probes combining a quencher and a fluorophore, said fluorescence probes being configured to each target a distinct nucleic acid sequence and said fluorophores emitting in overlapping fluorescence wavelengths;
b) during each thermal cycle, carrying out at least two fluorescence measurements in said fluorescence wavelength ranges, the radiation emitted by each probe varying depending on whether the probe is in a non-modified state in which the quencher attenuates or does not attenuate substantially the fluorescence emission, or in a modified state in which the quencher has an opposite effect on the fluorescence emission;
c) determining a value representative of the concentration of each fluorescence probe in a modified state from the measurements of operation b) and from time signatures derived from the measured fluorescence as a function of time for a given thermal cycle of a reaction mixture comprising one or more probes, which may each be substantially entirely in a non-modified or modified state, said values representative of a concentration in a modified state making it possible to qualify the presence of one or more nucleic acid sequences each associated with a distinct fluorescence probe so as to cause the fluorescence probe to change from the non-modified state to the modified state when they interact.

10. The method according to claim 9, wherein the operation b) comprises carrying out PCR cycles, and the at least two fluorescence probes are selected from the group comprising TaqMan probes, molecular beacons, dual fluorescence transfer probes, Scorpion probes, Amplifluor probes, MGB Eclipse probes, LUX probes, QUASR probes and QZyme probes.

11. The method according to claim 9, wherein the operation b) comprises the use of an in vitro nucleic acid amplification reagent comprising three fluorescence probes combining a quencher and a fluorophore, said fluorescence probes being configured to each target a distinct nucleic acid sequence and said fluorophores emitting in overlapping fluorescence wavelengths.

12. The method according to claim 9, wherein the operation b) comprises the use of an in vitro nucleic acid amplification reagent comprising at least two groups of fluorescence probes combining a quencher and a fluorophore, said fluorescence probes being configured to each target a distinct nucleic acid sequence and said fluorophores emitting in overlapping fluorescence wavelengths within each group of fluorescence probes.

13. The method according to claim 9, wherein the operation c) comprises solving a matrix system based on the fluorescence measurements, the time signatures and the constancy of the respective concentration of each fluorescence probe.

14. The method according to claim 12, wherein the operation c) comprises solving one matrix system per group of fluorescence probes.

15. The method according to claim 9, wherein the operation c) comprises applying a gradient descent or using a neural network.
